(19) United States
(12) Patent Application Publication
(10) Pub. No.: US 2018/0168501 A1
(43) Pub. Date: Jun. 21, 2018

(54) SYSTEMS AND METHODS FOR PERFORMING NERVE MONITORING

(71) Applicant: Jack M. Kartush, Bloomfield Hills, MI (US)

(72) Inventor: Jack M. Kartush, Bloomfield Hills, MI (US)

(21) Appl. No.: 15/816,033

(22) Filed: Nov. 17, 2017

Related U.S. Application Data
(60) Provisional application No. 62/435,914, filed on Dec. 19, 2016.

(51) Int. Cl. A61B 5/00; A61N 1/36; A61N 1/05

(52) U.S. Cl. CPC A61B 5/4519; A61B 5/6877; A61N 1/36114; A61B 2562/227; A61B 2560/0468; A61B 2562/164; A61N 1/0517

(57) ABSTRACT

This disclosure relates to systems and methods for performing nerve monitoring. An exemplary nerve monitoring system may include one or more stimulating devices for stimulating target nerves and one or more recording devices for recording responses from muscles associated with the target nerves. The methods can be employed for simultaneously monitoring the recurrent laryngeal nerve, the superior laryngeal nerve, and the vagus nerve during a surgical procedure.

### CROSS-REFERENCE TO RELATED APPLICATIONS

This application claims priority to U.S. Provisional Application No. 62/435,914, which was filed on Dec. 19, 2016.

### TECHNICAL FIELD

This disclosure relates to systems and methods for performing nerve monitoring. An exemplary nerve monitoring system may include a stimulating device for stimulating a vagus nerve and a recording device for recording electromyography responses elicited by stimulating a superior laryngeal nerve.

### BACKGROUND

Intraoperative nerve monitoring may be employed to reduce the incidence of laryngeal nerve injury during thyroid, skull base, cervical spine, or other surgeries. A common form of nerve monitoring uses electromyography (EMG) to detect the location of, and damage to, the laryngeal nerves. In such a technique, for example, intermittent electric stimulation of a target nerve is used to elicit laryngeal muscle contractions which are recorded by various recording devices, such as an endotracheal tube having one or more surface electrodes. An EMG response following stimulation of a target nerve confirms that the nerve has been located and is intact.

Although effective for some nerves, known nerve monitoring techniques have not proven reliable for detecting EMG responses elicited by stimulating the superior laryngeal nerve (SLN). Indeed, false monitoring errors are common when monitoring the SLN because of the lack of proximity of recording devices to the muscles associated with the SLN. In addition, the vagus nerve is not routinely monitored because of the difficulties associated with exposing the vagus nerve for direct stimulation. Accordingly, additional advances in this surgical field are desired.

### SUMMARY

This disclosure relates to systems and methods for performing nerve monitoring. An exemplary nerve monitoring system may include one or more stimulating devices for stimulating target nerves and one or more recording devices for recording responses from muscles associated with the target nerves.

Another exemplary nerve monitoring system includes a stimulating device configured to stimulate a vagus nerve without being in direct contact with the vagus nerve.

Another exemplary nerve monitoring system includes a recording device adapted to be affixed to a cricothyroid muscle and configured to record EMG responses from the cricothyroid muscle in response to stimulating the superior laryngeal nerve.

A nerve monitoring system according to another exemplary aspect of the present disclosure includes, among other things, a stimulating device configured to stimulate a vagus nerve, a first recording device configured to record electromyography responses from a muscle associated with the vagus nerve, and an electrical probe configured to stimulate a superior laryngeal nerve. A second recording device is adapted to be affixed to a cricothyroid muscle and is configured to record electromyography responses from the cricothyroid muscle in response to stimulating the superior laryngeal nerve.

In a further non-limiting embodiment of the foregoing system, the stimulating device stimulates the vagus nerve without directly contacting the vagus nerve.

In a further non-limiting embodiment of either of the foregoing systems, the stimulating device includes a flexible carrier having an internal concave side and an external convex side. The internal concave side is curved to conform to an outer convexity of a carotid sheath that surrounds the vagus nerve.

In a further non-limiting embodiment of any of the foregoing systems, at least one electrode is mounted to the internal concave side of the flexible carrier.

In a further non-limiting embodiment of any of the foregoing systems, a wire connects between the flexible carrier and an electrical stimulus generator.

In a further non-limiting embodiment of any of the foregoing systems, the external convex side of the flexible carrier includes barbs, hooks, or flexible strands.

In a further non-limiting embodiment of any of the foregoing systems, the flexible carrier is made of a polymeric material.

In a further non-limiting embodiment of any of the foregoing systems, the second recording device includes a flexible carrier having an internal concave side and an external convex side. The internal concave side is curved to conform to an outer convexity of the cricothyroid muscle.

In a further non-limiting embodiment of any of the foregoing systems, at least one electrode is mounted to the internal concave side of the flexible carrier.

In a further non-limiting embodiment of any of the foregoing systems, a wire extends from the flexible carrier to an electromyography monitoring device.

In a further non-limiting embodiment of any of the foregoing systems, the flexible carrier is made of a polymeric material.

In a further non-limiting embodiment of any of the foregoing systems, the first recording device includes an endotracheal tube and at least one electrode carried by the endotracheal tube.

In a further non-limiting embodiment of any of the foregoing systems, the electrical probe is configured to apply an electrical impulse or voltage to the superior laryngeal nerve.

A method for performing nerve monitoring according to another exemplary aspect of the present disclosure includes, among other things, positioning a stimulating device on an outer convexity of a carotid sheath, positioning a recording device within a laryngeal space, stimulating a vagus nerve with the stimulating device, and recording an electromyography response of a muscle associated with the vagus nerve with the recording device.

In a further non-limiting embodiment of the foregoing method, the stimulating device stimulates the vagus nerve without directly contacting the vagus nerve.

In a further non-limiting embodiment of either of the foregoing methods, the method includes positioning a second recording device relative to an outer convexity of a cricothyroid muscle, stimulating a superior laryngeal nerve with an electrical probe, and recording an electromyography response of the cricothyroid muscle with the second recording device in response to stimulating the superior laryngeal nerve.

In a further non-limiting embodiment of any of the foregoing methods, the method includes stimulating a recurrent laryngeal nerve with an electrical probe and recording an electromyography response of the recurrent laryngeal nerve with the recording device.

In a further non-limiting embodiment of any of the foregoing methods, the method includes positioning a recording device relative to an outer convexity of a cricothyroid muscle, stimulating a superior laryngeal nerve with an electrical probe, and recording an electromyography response of the cricothyroid muscle with the recording device in response to stimulating the superior laryngeal nerve.

In a further non-limiting embodiment of any of the foregoing methods, the method includes positioning a stimulating device on an outer convexity of a carotid sheath, positioning a second recording device within a laryngeal space, stimulating a vagus nerve with the stimulating device, and recording an electromyography response of a muscle associated with the vagus nerve with the second recording device.

In a further non-limiting embodiment of any of the foregoing methods, the method includes stimulating a recurrent laryngeal nerve with the electrical probe and recording an electromyography response of the recurrent laryngeal nerve with the second recording device.

Another exemplary method for performing nerve monitoring includes positioning a stimulating device on an outer convexity of a carotid sheath, positioning a recording device within a laryngeal space, stimulating a vagus nerve with the stimulating device, and recording an EMG response of a muscle associated with the vagus nerve with the recording device.

Another exemplary method for performing nerve monitoring includes positioning a recording device relative to an outer convexity of a cricothyroid muscle, stimulating a superior laryngeal nerve with an electrical probe, and recording an EMG response of the cricothyroid muscle with the recording device in response to stimulating the superior laryngeal nerve.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 schematically illustrates the anatomy of a human neck.

FIG. 2 illustrates a nerve monitoring system according to an exemplary embodiment of this disclosure.

FIGS. 3 and 4 illustrate a stimulating device of the nerve monitoring system of FIG. 2.

FIG. 5 illustrates an electrode arrangement of the stimulating device of FIGS. 3 and 4.

FIGS. 6A, 6B and 6C illustrate various attachment features of the stimulating device of FIGS. 3 and 4.

FIGS. 7 and 8 illustrate a recording device of the nerve monitoring system of FIG. 2.

FIG. 9 illustrates an electrode arrangement of the recording device of FIGS. 7 and 8.

FIG. 10 schematically illustrates a method of performing nerve monitoring according to an exemplary embodiment of this disclosure.

### DETAILED DESCRIPTION

This disclosure describes systems and methods for performing nerve monitoring. An exemplary nerve monitoring system includes one or more stimulating devices for stimulating nerves and one or more recording devices for recording electromyography responses elicited by stimulating the nerves. These and other features of this disclosure are discussed in greater detail below.

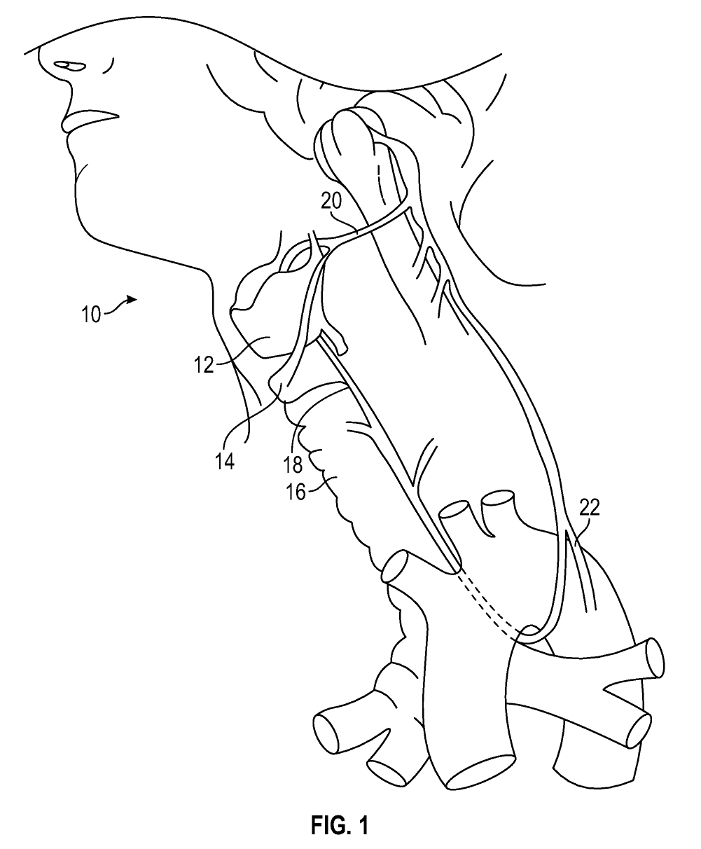

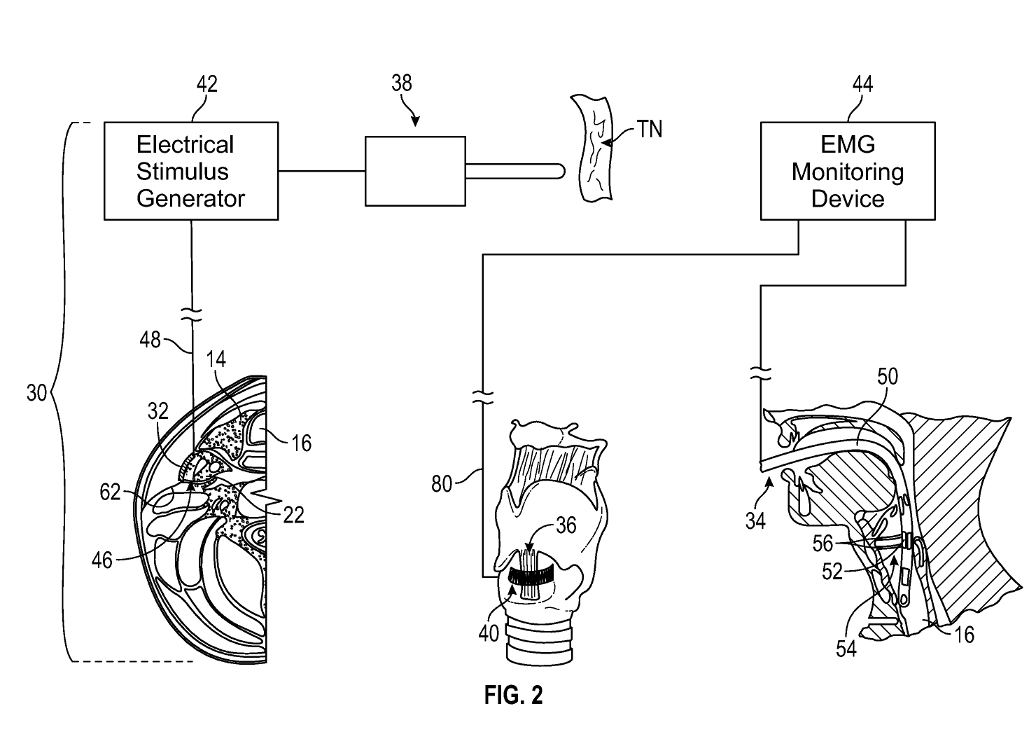

FIG. 1 illustrates the anatomy of a human neck 10. Among various other anatomical structures, the human neck 10 includes a larynx 12, a thyroid gland 14, a trachea 16, and various nerves. The nerves which are relevant for the purposes of this disclosure include the recurrent laryngeal nerve (RLN) 18, the superior laryngeal nerve (SLN) 20, and the vagus nerve 22. The RLN 18 innervates a majority of the intrinsic muscles of the larynx 12 and carries sensory information from the larynx. The external branch of the SLN 20 innervates the cricothyroid muscle (see feature 36 of FIG. 2), which increases tension of the vocal cords allowing higher, sustained pitches, and the internal branch of the SLN 20 provides sensory information from the superior larynx. Finally, the vagus nerve 22 is responsible for numerous tasks including but not limited to innervating portions of the larynx and the heart.

It may be desirable to monitor one or more of the RLN 18, the SLN 20 and the vagus nerve 22 for identifying and avoiding damage to these nerves during surgery, such as during thyroid, skull base, or cervical spine surgeries, for example. This disclosure describes systems and methods for simultaneously monitoring the RLN 18, the SLN 20, and the vagus nerve 22 during a surgical procedure.

FIG. 2, with continued reference to FIG. 1, illustrates an exemplary nerve monitoring system 30. In a non-limiting embodiment, the nerve monitoring system 30 may be used to perform intraoperative nerve monitoring of each of the RLN 18, the SLN 20, and the vagus nerve 22 during various surgical procedures. The nerve monitoring system 30 may include a stimulating device 32 for stimulating the vagus nerve 22, a first recording device 34 for recording EMG responses elicited by stimulating the RLN 18 and/or the vagus nerve 22, an electrical probe 38 for stimulating the SLN 20 to elicit an EMG response of a cricothyroid muscle 36, a second recording device 40 for recording EMG responses from the cricothyroid muscle 36, an electrical stimulus generator 42, and an EMG monitoring device 44, from which the stimulus is typically generated and the response recorded.

The stimulating device 32 is adapted to conform to an outer convexity of the carotid sheath 46, within which the vagus nerve 22 is situated. The stimulating device 32 may include a wire 48 that extends to connect to the electrical stimulus generator 42. An output current of the electrical stimulus generator 42 may be controlled to power one or more electrodes of the stimulating device 32 in order to stimulate the vagus nerve 22 and thus elicit an EMG response from vocal muscles associated with the vagus nerve 22 as a way to supplement monitoring of the RLN 18.

The stimulating device 32 allows stimulation of the vagus nerve 22 without the need to dissect the carotid sheath 46. In other words, direct contact with the vagus nerve 22 is not required to stimulate the vagus nerve 22. The stimulating device 32 thus provides “near-field, non-contact” vagal stimulation that reduces risks to the vagus nerve 22, the carotid artery, and the jugular vein as compared to techniques currently utilized in this surgical field which require opening the carotid sheath 46 to expose the vagus nerve 22 for direct stimulation.

The first recording device 34 records EMG responses elicited by stimulating both the RLN 18 and the vagus nerve 22. For example, the first recording device 34 may include an endotracheal tube 50 that may be inserted through a patient's nose or mouth and into the trachea 16. One or more electrodes 52 are mounted to or otherwise carried by the endotracheal tube 50. The endotracheal tube 50 may be positioned within a laryngeal space 54 such that the electrode(s) 52 are aligned with the vocal cords 56, in a non-limiting embodiment.

Once the first recording device 34 has been properly positioned, a surgeon may move the electrical probe 38 to a location where the target nerve TN (e.g., the RLN 18, etc.) is suspected to be located. The electrical probe 38 provides an intermittent or constant electrical impulse or voltage, generated by the electrical stimulus generator 42, which in turn is transmitted through the target nerve TN and then to an associated target muscle, such as the vocal cords 56. The electrical impulse which is carried through the target nerve TN causes the target muscles to contract and generate their own electric pulses. The EMG response of the target muscle is measured or otherwise sensed by the electrodes 52 of the first recording device 34. These measurements are outputted to the EMG monitoring device 44, which provides an audible or visual alert if the electrical probe 38 is in proximity to the target nerve TN. Similarly, the first recording device 34 can measure or otherwise sense activity of the target muscles in response to stimulation of the vagus nerve 22 via the stimulating device 32. In this way, the surgeon can perceive the location of the target nerve TN and avoid further activity in that area to prevent damaging the target nerve TN.

The electrical probe 38 may additionally be used to stimulate the SLN 20, such as by applying an electrical impulse or voltage to the external branch of the SLN 20 at a location near the superior pole of the thyroid. The electrical impulse provided by the electrical probe 38 travels through the SLN 20 to the cricothyroid muscle 36. The second recording device 40 records EMG responses elicited by stimulating the SLN 20. For example, the cricothyroid muscle 36 contracts and generates its own electric pulses in response to stimulating the SLN 20. These electric pules are measured or otherwise sensed by the second recording device 40. These measurements are outputted to the EMG monitoring device 44, which provides an audible or visual alert if the electrical probe 38 is in proximity to the SLN 20, thus permitting the surgeon to perceive the location of the SLN 20 and avoid damaging the SLN 20 during surgery.

The second recording device 40 may advantageously be positioned intramuscularly. It therefore provides superior, muscle-specific recording compared to relying on endotracheal electrodes that are adjacent to most vocal cord muscles but still relatively remote from the cricothyroid muscle 36 and can thus result in false negative monitoring errors when attempting to locate the SLN 20.

The target nerve TN may be electrically stimulated either intermittently or continuously. In a non-limiting embodiment, the RLN 18 and the SLN 20 are intermittently stimulated using the electrical probe 38, and the vagus nerve 22 is continuously stimulated using the stimulating device 32. The target nerve TN could also be stimulated by surgical manipulation, trauma, thermal changes, etc.

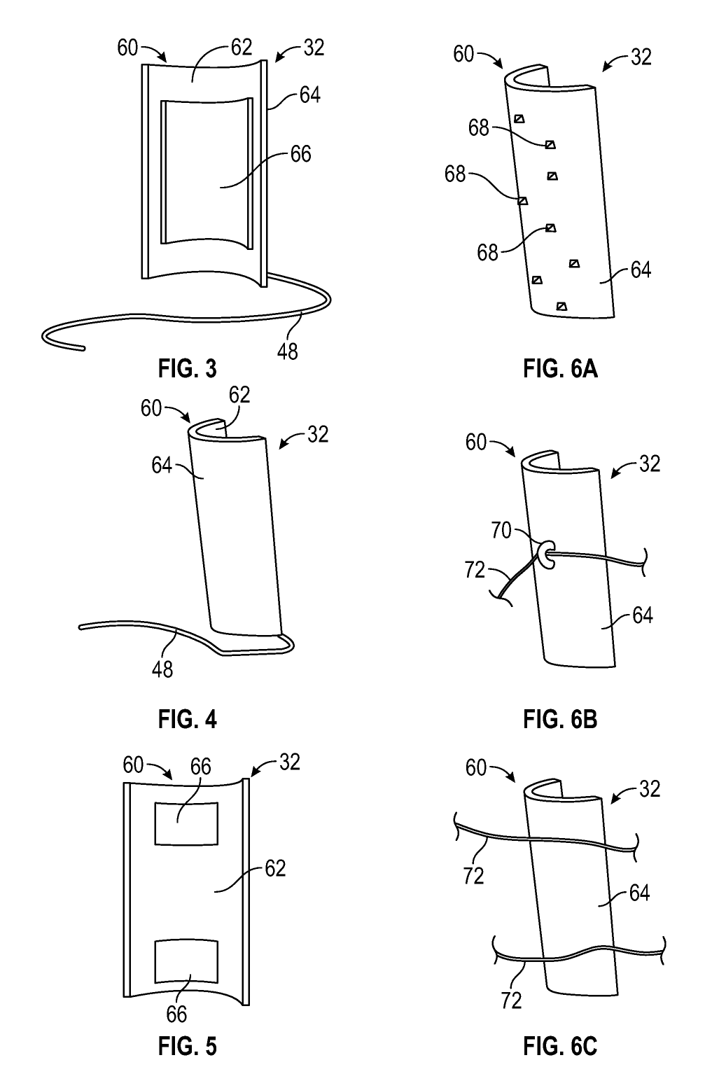

FIGS. 3 and 4 illustrate additional details of the stimulating device 32 of the nerve monitoring system 30 of FIG. 2. The stimulating device 32 includes a flexible carrier 60 having an internal concave side 62 and an external convex side 64. The internal concave side 62 is curved to conform to the outer convexity of the carotid sheath 46 (see FIG. 2, for example). A wire 48 extends from the flexible carrier 60 for connecting to the electrical stimulus generator 42 (see FIG. 2).

In a non-limiting embodiment, the flexible carrier 60 is made of a polymeric material which provides the flexible characteristics of the stimulating device 32. In another non-limiting embodiment, the flexibility of the stimulating device 32 can be achieved by providing one or more hinged joints on the flexible carrier 60. The flexibility of the flexible carrier 60 aids in conforming the stimulating device 32 to the outer convexity of the carotid sheath 46.

One or more electrodes 66 may be mounted to or integrated with the internal concave side 62 of the flexible carrier 60. The electrodes 66 are configured to provide an electrical impulse or voltage for continuously stimulating the vagus nerve 22, in a non-limiting embodiment. The stimulating device 32 may include a single electrode 66 (see FIG. 3) or multiple electrodes (see FIG. 5). For example, FIG. 5 illustrates a bipolar configuration. It should be understood that the stimulating device 32 is not limited to the specific configurations shown in FIGS. 2 through 5 and could include any amount of electrodes arranged in any configuration.

The external convex side 64 of the flexible carrier 60 may be a smooth surface. In other embodiments, the external convex side 64 of the stimulating device 32 may include various attachment features for stabilizing the stimulating device 32 relative to surrounding soft tissue, such as tissue that is adjacent to the carotid sheath 46. Referring first to FIG. 6A, for example, the external convex side 64 of the flexible carrier 60 may include a plurality of barbs 68. The barbs 68 jut outwardly from the external convex side 64 and can grip tissue once the stimulating device 32 is positioned relative to the carotid sheath 46.

In another non-limiting embodiment, shown in FIG. 6B, the external convex side 64 of the flexible carrier 60 may include one or more hooks 70. A flexible strand 72, such as suture, may be threaded through the hooks 70 and then tied to tissue to stabilize the stimulating device 32 once positioned relative to the carotid sheath 46. In yet another non-limiting embodiment, shown in FIG. 6C, one or more flexible strands 72 are integrally molded with or otherwise directly attached to the external convex side 64 of the flexible carrier 60.

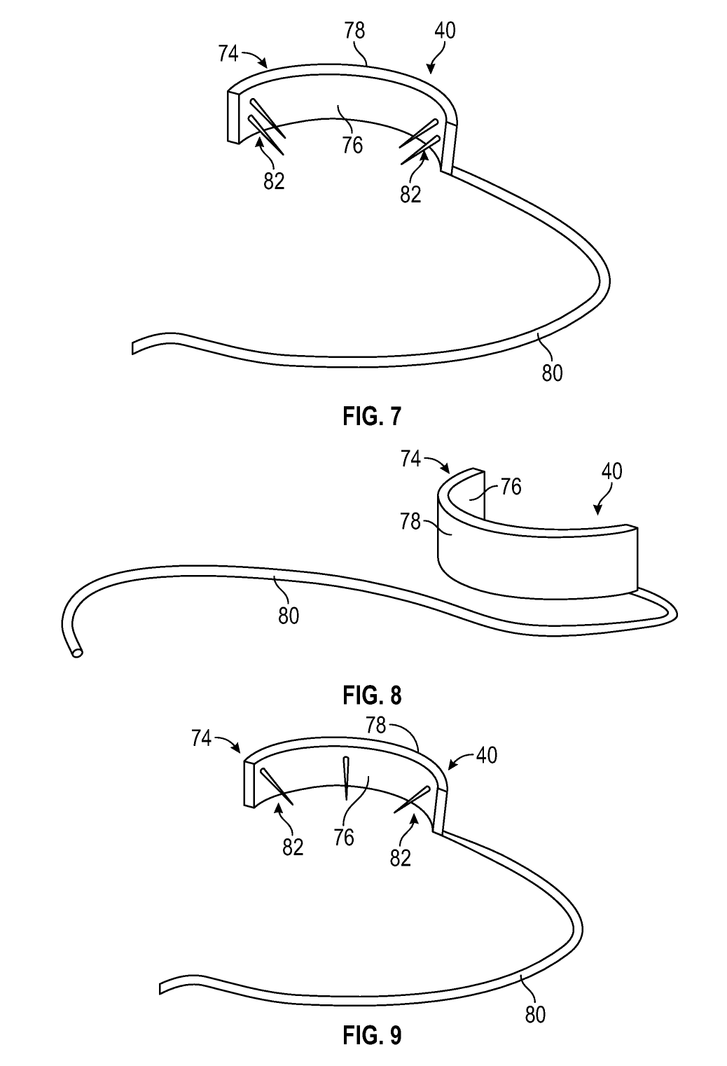

FIGS. 7-8 illustrate additional features of the second recording device 40 of the nerve monitoring system 30 of FIG. 2. The second recording device 40 includes a flexible carrier 74 having an internal concave side 76 and an external convex side 78. The internal concave side 76 is curved to conform to the outer convexity of the cricothyroid muscle 36 (see FIG. 2, for example), and the external convex side 78 may be a generally smooth surface. A wire 80 extends from the flexible carrier 74 for connecting to the EMG monitoring device 44 (see FIG. 2). The flexible carrier 74 may be made of a polymeric material and/or could include one or more hinged joints for imparting flexibility.

One or more electrodes 82 may protrude from the internal concave side 76 of the flexible carrier 74. The electrodes 82 are configured to measure or otherwise sense EMG responses from the cricothyroid muscle 36, in a non-limiting embodiment. In another non-limiting embodiment, the electrodes 82 are needle electrodes that may be inserted bilaterally into a target muscle for securely attaching the second recording device 40 to the target muscle. The second recording device 40 may include two bipolar pairs of electrodes 82 (FIG. 7) or could include a single pair of monopolar electrodes 82 (FIG. 9). It should be understood that the second recording device is not limited to the specific configurations shown in FIGS. 7-9 and could include any amount of electrodes arranged in any configuration.

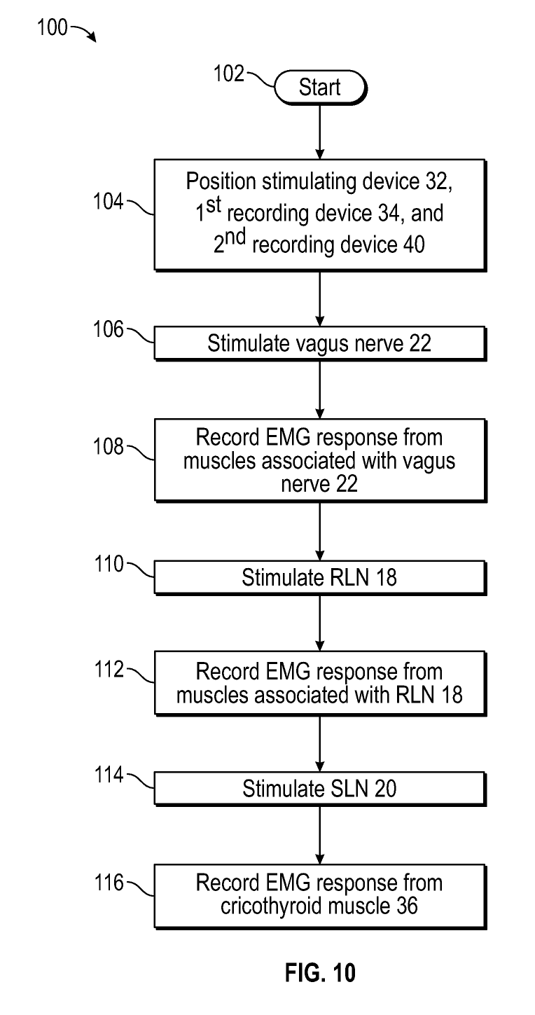

FIG. 10, with continued reference to FIGS. 1-9, schematically illustrates an exemplary method 100 for performing nerve monitoring using the nerve monitoring system 30 of FIG. 2. It should be understood that the method described herein may include a greater or fewer number of steps and that the steps could be performed in a different order within the scope of this disclosure.

The method 100 begins at block 102. At block 104, the stimulating device 32, the first recording device 34, and the second recording device 40 are positioned at desired locations of a patient prepped to undergo surgery. For example, the stimulating device 32 may be mounted relative to the outer convexity of the carotid sheath 46, the first recording device 34 may be positioned within the laryngeal space 54, and the second recording device 40 may be positioned relative to the cricothyroid muscle 36.

Next, at block 106, the vagus nerve 22 may be stimulated using the stimulating device 32. The first recording device 34 records an EMG response of a muscle associated with the vagus nerve 22 (e.g., the vocal cords 56 and associated muscles) at block 108. Because both the RLN 18 and the SLN 20 come off as branches from the vagus nerve 22, a response from the main vocal cord muscles can occur when stimulating the vagus nerve 22. Thus, the second recording device 40 may also record EMG responses in response to stimulating the vagus nerve 22 because some of the electrical impulses are going to the SLN 20 and then to the cricothyroid muscle 36. In another embodiment, both the first recording device 34 and the second recording device 40 record the EMG responses.

The RLN 18 may be stimulated at block 110 by using the electrical probe 38. The first recording device 34, the second recording device 40, or both records an EMG response of a muscle associated with the RLN 18 (e.g., the vocal cords 56 and associated muscles) at block 112.

At block 114, the SLN 20 is stimulated by using the electrical probe 38. The second recording device 40 records an EMG response of the cricothyroid muscle 36 at block 116. The exemplary method 100 thus describes a novel technique for simultaneously monitoring the RLN 18, the SLN 20, and the vagus nerve 22 during a surgical procedure.

The exemplary nerve monitoring systems and methods of this disclosure are effective for monitoring the SLN in addition to the RLN. By positioning recording devices intramuscularly when monitoring the SLN, the nerve monitoring system provides superior, muscle-specific recording of the cricothyroid muscle when stimulating the SLN. Moreover, the exemplary systems and methods provide the ability to stimulate the vagus nerve without the need to expose it for direct stimulation (i.e. near-field non-contact stimulation), therein reducing the potential for inadvertent surgical trauma to the vagus nerve, jugular vein, and carotid artery. The proposed systems and methods are therefore safer, more accurate, and improve medical outcomes as compared to known techniques.

Although the different non-limiting embodiments are illustrated as having specific components, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A nerve monitoring system, comprising:
a stimulating device configured to stimulate a vagus nerve;
a first recording device configured to record electromyography responses from a muscle associated with the vagus nerve;
an electrical probe configured to stimulate a superior laryngeal nerve; and
a second recording device adapted to be affixed to a cricothyroid muscle and configured to record electromyography responses from the cricothyroid muscle in response to stimulating the superior laryngeal nerve.

2. The system as recited in claim 1, wherein the stimulating device stimulates the vagus nerve without directly contacting the vagus nerve.

3. The system as recited in claim 1, wherein the stimulating device includes a flexible carrier having an internal concave side and an external convex side, and the internal concave side is curved to conform to an outer convexity of a carotid sheath that surrounds the vagus nerve.

4. The system as recited in claim 3, comprising at least one electrode mounted to the internal concave side of the flexible carrier.

5. The system as recited in claim 3, comprising a wire that connects between the flexible carrier and an electrical stimulus generator.

6. The system as recited in claim 3, wherein the external convex side of the flexible carrier includes barbs, hooks, or flexible strands.

7. The system as recited in claim 3, wherein the flexible carrier is made of a polymeric material.

8. The system as recited in claim 1, wherein the second recording device includes a flexible carrier having an internal concave side and an external convex side, and the internal concave side is curved to conform to an outer convexity of the cricothyroid muscle.

9. The system as recited in claim 8, comprising at least one electrode mounted to the internal concave side of the flexible carrier.

10. The system as recited in claim 8, comprising a wire that extends from the flexible carrier to an electromyography monitoring device.

11. The system as recited in claim 8, wherein the flexible carrier is made of a polymeric material.

12. The system as recited in claim 1, wherein the first recording device includes an endotracheal tube and at least one electrode carried by the endotracheal tube.

13. The system as recited in claim 1, wherein the electrical probe is configured to apply an electrical impulse or voltage to the superior laryngeal nerve.

14. A method for performing nerve monitoring, comprising:
positioning a stimulating device on an outer convexity of a carotid sheath;
positioning a recording device within a laryngeal space;
stimulating a vagus nerve with the stimulating device; and
recording an electromyography response of a muscle associated with the vagus nerve with the recording device.

15. The method as recited in claim 14, wherein the stimulating device stimulates the vagus nerve without directly contacting the vagus nerve.

16. The method as recited in claim 14, comprising:
positioning a second recording device relative to an outer convexity of a cricothyroid muscle;
stimulating a superior laryngeal nerve with an electrical probe; and
recording an electromyography response of the cricothyroid muscle with the second recording device in response to stimulating the superior laryngeal nerve.

17. The method as recited in claim 14, comprising:
stimulating a recurrent laryngeal nerve with an electrical probe; and
recording an electromyography response of the recurrent laryngeal nerve with the recording device.

18. A method for performing nerve monitoring, comprising:
positioning a recording device relative to an outer convexity of a cricothyroid muscle;
stimulating a superior laryngeal nerve with an electrical probe; and
recording an electromyography response of the cricothyroid muscle with the recording device in response to stimulating the superior laryngeal nerve.

19. The method as recited in claim 18, comprising:
positioning a stimulating device on an outer convexity of a carotid sheath;
positioning a second recording device within a laryngeal space;
stimulating a vagus nerve with the stimulating device; and
recording an electromyography response of a muscle associated with the vagus nerve with the second recording device, the recording device, or both.

20. The method as recited in claim 19, comprising:
stimulating a recurrent laryngeal nerve with the electrical probe; and
recording an electromyography response of the recurrent laryngeal nerve with the second recording device, the recording device, or both.
